(19) United States
(12) Patent Application Publication
(10) Pub. No.: US 2026/0317004 A1
(43) Pub. Date: Oct. 15, 2026

(54) Conforming and Compressing Packaging System

(71) Applicant: Howmedica Osteonics Corp., Mahwah, NJ (US)

(72) Inventor: Mark Kansburg, Hewitt, NJ (US)

(73) Assignee: Howmedica Osteonics Corp., Mahwah, NJ (US)

(21) Appl. No.: 19/463,350

(22) Filed: Jan. 29, 2026

Related U.S. Application Data
(60) Provisional application No. 63/751,902, filed on Jan. 31, 2025.

(51) Int. Cl. B65D 25/08; B65D 25/10

(52) U.S. Cl. CPC B65D 25/08; B65D 25/10

(57) ABSTRACT

A packaging device includes a first insert that has a rim enclosing a recessed portion. The recessed portion includes a base and a wall extending between the rim and the base, the wall having a plurality of steps. The first insert is configured such that the rim is receivable on a surface of any one container from among a plurality of containers, where the plurality of containers include a first container and a second container such that a first interior volume is defined between the first insert and a base of the first container when the first insert is received on the first container and a second interior volume is defined between the first insert and a base of the second container when the first insert is received on the second container, the first interior volume being different from the second interior volume.

### CROSS-REFERENCE TO RELATED APPLICATION

This application claims the benefit of the filing date of U.S. Provisional Patent Application No. 63/751,902, filed Jan. 31, 2025, the disclosure of which is hereby incorporated by reference herein in its entirety.

### BACKGROUND

In many instances, packaging used to store and transport objects may be subject to forces that may cause such objects to be damaged. Accordingly, objects that are sensitive to being exposed to such conditions, such as medical implants, are packaged to protect the object from damage. However, existing packages, particularly those configured to provide for the containment of objects with different shapes and/or sizes, may not prevent movement of the object inside the package. Further, some packaging utilizes semi-rigid foams to hold an object, but such foams have limited capacity for compression and may generate particulate when used in packaging or may cause an enclosed implant to generate particulate, which may be deleterious to the preservation of the object for its intended use. To the extent any packaging is shaped to conform to an object, such packaging is cumbersome in that each object must be provided with unique custom packaging.

Accordingly, there is a need for packaging that better secures an object against movement within packaging and that reduces the amount of packaging components required to be able to individually hold each object among a variety of objects.

### BRIEF SUMMARY

The present disclosure contemplates packaging systems, devices, kits and associated methods that advantageously protect an object stored by such packaging against movement within the packaging and any damage that would otherwise result from such movement. The packaging contemplated by the present disclosure also reduces the number of packaging components needed to contain a collection of objects having a variety of shapes compared to packaging designed to contain an object with a predetermined size and shape. Examples of objects that may be stored by the contemplated packaging and packaging components include medical implants, for example.

In a first aspect, the present disclosure relates to a packaging device. In a first example of a first embodiment, a packaging device includes a first insert that has a rim that encloses a recessed portion. The recessed portion includes a base and a wall extending between the rim and the base, the wall having a plurality of steps. The first insert is configured such that the rim is receivable on a surface of any one container from among a plurality of containers. Further, the plurality of containers include a first container and a second container such that a first interior volume is defined between the first insert and a base of the first container when the first insert is received on the first container and a second interior volume is defined between the first insert and a base of the second container when the first insert is received on the second container, the first interior volume being different from the second interior volume.

In a second example of the first embodiment, the packaging device of the first example may be configured such that each step of the plurality of steps is separated by a wall portion of a plurality of wall portions, each of the wall portions having a varying radius of curvature along its length. In a third example, the packaging device of the first or second example may be configured such that a portion of the wall of the first insert that includes the plurality of steps is deformable when placed over and pressed against an object disposed in one container from among the plurality of containers. In a fourth example, the packaging device of any one of the first through third examples may be configured such that the plurality of steps on the wall include edges having a varying radius of curvature along their respective lengths. In a fifth example, the packaging device of any one of the first through fourth examples may be configured such that the first insert includes a plurality of radial protrusions extending from the base of the recessed portion to the rim along the wall of the recessed portion, the radial protrusions being on an outer surface of the recessed portion opposite an inner surface of the recessed portion that defines a cavity. In a sixth example, the packaging device of any one of the first through fifth examples may include the first container from among the plurality of containers.

In a seventh example, the packaging device of any one of the first through sixth examples may be part of a packaging system that also includes a second insert, the second insert being receivable on a bottom surface of any one container from among the plurality of containers. In an eighth example, the packaging system of the seventh example may be configured such that the second insert includes a plurality of protrusions thereon, the plurality of protrusions being adapted to resist movement of an object disposed thereon.

In a second embodiment, a packaging device may be an insert configured to restrain an object when the object is disposed in a container. In a first example, the packaging device includes an insert having a rim and a recessed portion within the rim, the recessed portion including a wall and a base where the wall extends from the rim to the base. The wall of the recessed portion includes a plurality of wall portions each separated by a step of a plurality of steps, each wall portion extending along the wall circumferentially around a central axis of a cavity defined by the wall and the base, the central axis passing through a center of the base and a center of an opening defined by the rim. At least one wall portion from among the plurality of wall portions has an outer surface with a first segment having a convex surface and a second segment having a concave surface.

In a second example of the second embodiment, the insert of the first example may be configured such that the wall includes a plurality of radial protrusions thereon, each radial protrusion of the plurality of radial protrusions being spaced apart from the others and extending from the base to the rim. In a third example, the insert of the second example may be configured such that the plurality of radial protrusions includes four radial protrusions, each radial protrusion of the four radial protrusions being raised relative to the plurality of wall portions. In a fourth example, the insert of any one of the first through third examples may be configured such that the rim includes an upper surface transverse to the central axis, the upper surface including a peripheral edge with a peripheral recess, the peripheral recess having a concave contour over a majority of its length.

In a second aspect, the present disclosure relates to a packaging system. In a first example of a fist embodiment, a packaging system includes a first insert and a second insert. The first insert includes a recessed portion extending from an enclosed rim, the recessed portion having a central axis passing through a pole of the recessed portion and a center of an opening defined by the enclosed rim such that each step of a plurality of steps on the recessed portion extends circumferentially around the central axis. The second insert includes a plurality of protrusions arranged around a central location of the second insert. The first and second inserts are configured to be received on respective first and second surfaces of a container. When the second insert is disposed on the second surface of the container and an object is disposed on the second insert, the disposal of the first insert onto the object causes the first insert to deform as the first insert is received on the first surface of the container. And, when the first insert, the second insert, and the object are received in the container, the first and second inserts restrain the object from movement relative to the first and second inserts.

In a second example of the first embodiment, the packaging system of the first example may be configured such that the first insert includes a plurality of openings therethrough. In a third example, the packaging system of any one of the first and second examples may be configured such that the first insert includes a plurality of radially extending portions that extend from a base of the first insert toward the enclosed rim. In a fourth example, the packaging system of the third example may be configured such that each radially extending portion of the plurality of radially extending portions is raised relative to the plurality of steps. In a fifth example, the packaging system of the fourth example may be configured such that at least one radially extending portion of the plurality of radially extending portions includes an opening therethrough. In a sixth example, the packaging system of any one of the first through fifth examples may be configured such that each step of the plurality of steps is separated by a wall portion of a plurality of wall portions, each wall portion having a varying radius of curvature along its length. In a seventh example, the packaging system of the sixth example may be configured such that each step of the plurality of steps includes an outer surface configured to face the second insert when the first and second inserts are received in the container, the outer surface having a varying curvature along its length such that a centerline of the outer surface alternates between moving closer to and further from the central axis along its length.

In an eighth example of the first embodiment, the packaging system of any one of the first through seventh examples may be configured such that when the first insert, the second insert, and the object are received in the container and enclosed by a lid secured to the container, the first and second inserts restrain the object from movement relative to the first and second inserts. In a ninth example, the packaging system of any one of the first through seventh examples may include a lid configured to receive the first insert such that when the first insert is disposed onto the object in the container, the lid with the first insert secured thereto is secured to the container to enclose the object within the container.

In a third aspect, the present disclosure relates to a kit including components for packaging an object. In a first example of a first embodiment, the kit includes a first insert, a second insert, a first container and a second container. The first insert includes a rim enclosing a recessed portion. The second insert includes a surface with a base portion and a receiving portion, the receiving portion being raised relative to the base portion. The first container includes a first receiving portion defining a first cavity, the first container having a first base surface configured to receive the second insert and a first wall surface separate from the first base surface configured to receive the first insert. The second container includes a second receiving portion defining a second cavity, the second container having a second base surface configured to receive the second insert and a second wall surface separate from the second base surface configured to receive the first insert. A volume of the first cavity is different from a volume of the second cavity.

In a second example of the first embodiment, the kit of the first example may be configured such that the first container has a first depth from a first container rim to a first container base and the second container has a second depth from a second container rim to a second container base, the first depth being different from the second depth. In a third example, the kit of the first or second example may be configured such that the base portion of the second insert includes a peripheral portion defined by plurality of slits such that when the first base receives the second insert, the peripheral portion is flat relative to a remainder of the base portion and when the second base receives the second insert, the peripheral portion is folded relative to the remainder of the base portion. In a fourth example, the kit of any one of the first through third examples may be configured such that the first container includes a lid adapted to enclose the first cavity, the lid including at least one opening so that when the lid encloses the first cavity, the first cavity is in fluid communication with an environment external to the first cavity. In a fifth example, the kit of the fourth example may be configured such that the rim of the first insert includes a first plurality of peripheral recesses and the lid includes a second plurality of peripheral recesses complementary to the first plurality of recesses such that when the first insert is received in the first container and the lid encloses the first container, the first cavity is in fluid communication with an environment external to the first cavity via the first and second plurality of peripheral recesses.

In a sixth example of the first embodiment, the kit of any one of the first through fifth examples may be configured such that the recessed portion of the first insert includes a base and a wall extending between the rim and the base, the wall having a plurality of steps. In a seventh example, the kit of any one of the first through fifth examples may be configured such that the recessed portion of the first insert extends between the rim and a base and includes a first wall, a second wall and a step in between the first wall and the second wall, at least one of the first wall and the second wall having a first segment with a convex surface and a second segment with a concave surface. In an eighth example, the kit of any one of the first through fifth examples may be configured such that the recessed portion of the first insert extends between the rim and a base and includes an outer surface with a plurality of radial protrusions, each radial protrusion of the plurality of radial protrusions being spaced apart from the others and extending from the rim to the base. In a ninth example, the kit of the eighth example may be configured such that at least one radial protrusion of the plurality of radial protrusions includes an opening therethrough.

In a fourth aspect, the present disclosure relates to a method of packaging an object. In a first example of a first embodiment, a method of packaging an object includes: causing an object to be received on an inner insert disposed in a container; and causing an outer insert to be engaged to the container such that the outer insert is disposed over the object. When the outer insert engages with the container, the outer insert deforms and the object is restrained from movement relative to the inner insert and the outer insert.

In a second example of the first embodiment, the method of the first example may be performed such that when the outer insert engages with the container, a curved wall between steps on a recessed portion of the outer insert deforms, the curved wall including a convex surface and a concave surface. In a third example, the method of any one of the first and second examples may be performed with an outer insert configured such that the curved wall has an elongate dimension extending circumferentially around the recessed portion parallel to a rim of the outer insert. In a fourth example, the method of any one of the first through third examples may be performed such that when the outer insert engages with the container, a lid is positioned over the outer insert and engages with the container so that the outer insert is in between the lid and the object. In a fifth example, the method of any one of the first through third examples may be performed such that prior to causing the outer insert to be engaged with the container, a lid is secured onto the outer insert such that after the outer insert is engaged to the container, the outer insert is in between the lid and the object. In a sixth example, the method of any one of the first through fifth examples may include causing a sterilization procedure to be performed on the object disposed within the container, the sterilization procedure including fluid communication between a source of the sterilization procedure external to the container and a volume between the outer insert and the inner insert within the container.

### BRIEF DESCRIPTION OF THE DRAWINGS

A more complete appreciation of the subject matter of the present disclosure and of the various advantages thereof can be realized by reference to the following detailed description in which reference is made to the accompanying drawings in which:

FIG. 1 is a top view of a packaging system according to an embodiment of the present disclosure;

FIG. 2 is a side cross-sectional view of the packaging system of FIG. 1;

FIG. 3 is a top view of the packaging system of FIG. 1 with a lid removed;

FIG. 4 is a top view of the packaging system of FIG. 1 with a lid and an outer insert removed;

FIG. 5 is a first perspective view of the outer insert of the packaging system of FIG. 1;

FIG. 6 is a second perspective view of the outer insert of the packaging system of FIG. 1;

FIG. 7 is a perspective view of a packaging system according to an embodiment of the present disclosure;

FIG. 8 is a side cross-sectional view of the packaging system of FIG. 7;

FIG. 9 is a perspective view of a packaging system according to an embodiment of the present disclosure;

FIG. 10 is a side cross-sectional view of the packaging system of FIG. 9;

FIG. 11 is a perspective view of an outer insert of the packaging systems of FIGS. 7 and 9;

FIG. 12 is a perspective view of an inner insert of the packaging systems of FIGS. 7 and 9;

FIG. 13 is side view of the packaging system of FIG. 7 with a portion of the container removed and a first object stored within the system;

FIG. 14 is side view of the packaging system of FIG. 9 with a portion of the container removed and a second object stored within the system;

FIG. 15 is side view of the packaging system of FIG. 9 with a portion of the container removed and a third object stored within the system; and

FIG. 16 is a flow chart of a method of using a packaging system according to an embodiment of the present disclosure.

### DETAILED DESCRIPTION

As used herein unless stated otherwise, the terms “about,” “approximately,” “generally,” and “substantially” are intended to mean that slight deviations from absolute are included within the scope of the term so modified.

In one aspect, the present disclosure relates to a system that includes an outer insert and an inner insert that are configured to be received in a container to hold an object therein. For the sake of brevity, the embodiments described herein refer to objects in the form of medical implants. However, it is envisioned that any type of object of a size appropriate to fit in the container may be stored in the container and that such objects are not limited to those used for medical applications.

In one embodiment, a system includes an inner insert 130, shown in FIGS. 2 and 4, and an outer insert 150, shown in FIGS. 2, 3 and 5-6. Inner insert 130 includes an upper surface 134 and a lower surface (not shown) opposite the upper surface, upper surface 134 being generally planar. A peripheral edge 132 of upper surface 134 may optionally conform to a periphery of an inner base surface of a container, such as a periphery of inner base surface 112 of container 110. In this manner, inner insert 130 may cover an inner base of a container when disposed therein. Parallel to peripheral edge 132 and defining a secondary periphery of inner insert 130 are a plurality of slits 136 passing through inner insert 130. In the embodiment shown in FIG. 4, the plurality of slits 136 include eleven slits, each having an elongate dimension generally parallel to an adjacent portion of peripheral edge 132. In other examples, any number of slits may be included. Slits allow regions of inner insert 130 adjacent to peripheral edge 132 to be folded if desired, i.e., via a hinge-type action, which may be advantageous to accommodate certain container shapes. For instance, if a set of containers includes containers of different sizes, e.g., a set of containers where each container has the same upper rim, the same sidewall angle, but a different depth, a base of a cavity of the larger containers may have a smaller surface area than the smaller containers. In such cases, inner insert 130 may be folded to fit when used with the larger containers, even if the surface area of the base is smaller than in the smaller containers, thereby ensuring that the inner insert 130 may be used in a wide range of container sizes. Peripheral regions of inner insert 130 between plurality of slits 136 and peripheral edge 132 may be separated by cut outs to render it easier to fold such peripheral regions. For example, with inner insert 130 shown in FIG. 4, there are seven cut outs along the periphery, each having a triangular shape. In variations, a fold line of inner insert 130 may be defined by a different number of slits, slits having another shape, or through other features, such as perforations.

Further, inner insert 130 includes a plurality of support protrusions 138A-H located radially from a central region 135 of inner insert 130 and arranged at spaced apart intervals to form a circular type arrangement, as shown in FIG. 4. A side view of support protrusions 138B, 138E is shown in FIG. 2 and is representative of each of the plurality of support protrusions. Specifically, each support protrusion of the plurality of support protrusions 138A-H may have an outer surface that gradually rises and widens in a direction radially away from central region 135 of inner insert 130. Collectively, plurality of support protrusions 138A-H may define a shallow cup-type shape surrounding central region 135. Such shape is advantageous in that it provides grip against an implant received thereon to aid in the stabilization of the implant when the inserts 130, 150 are used in a container. In some variations, one or more support protrusions of the plurality of support protrusions 138A-H may include one or more gripping features thereon. In some examples of these variations, the gripping features may be projections on the outer surface of the support protrusions. Such projections may be elongate across the outer surface, a series of bumps on the outer surface, or have other characteristics to enhance a gripping effect. The plurality of support protrusions with gripping features enhance an ability to position various sizes of implants with rounded surfaces onto inner insert 130 so that each size of implant may be disposed in a stable manner and in a predictable position.

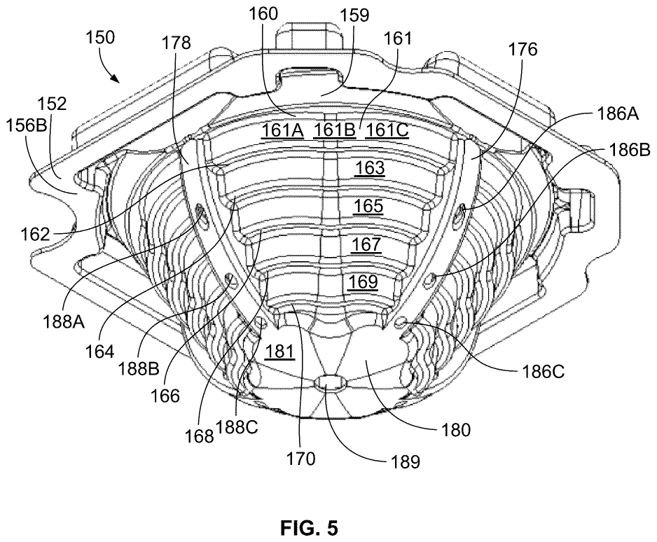

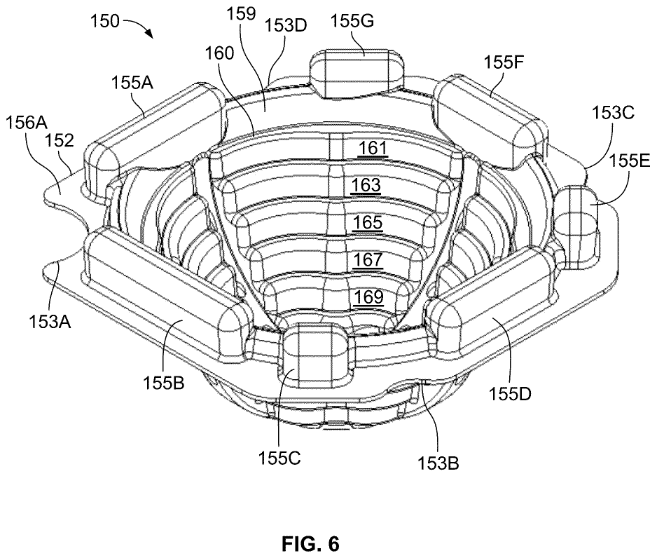

Outer insert 150, as shown in FIGS. 2, 3 and 5-6, is used in tandem with inner inset 130 to contain an object such as a medical implant within a container. With reference to FIGS. 5 and 6, outer insert 150 includes a rim 152 and a recessed portion extending from an inside of the rim. The recessed portion includes a wall extending from the rim and a base 180 enclosing the wall remote from rim 152. The wall includes a number of features as described in greater detail below. A combination of the wall and the base define a cavity within the recessed portion. In some examples, the recessed portion may have a central axis that passes through an apex of the base and a center of an opening defined by the rim.

As to rim 152, and with continued reference to FIGS. 5 and 6, rim 152 includes an upper surface 156A and a lower surface 156B. Rim 152 includes a peripheral edge with a plurality of peripheral recesses 153A-153D, each respective peripheral recess being in a plane aligned with upper and lower surfaces 156A-B of rim thereby narrowing a width of such upper and lower surfaces 156A-B. As described in greater detail elsewhere in the present disclosure, peripheral recesses 153A-153D function as openings when outer insert 150 is disposed in a container, such openings allowing for fluid such as gases to flow therethrough. Further, protruding from upper surface 156A of rim 152 are a plurality of rim protrusions 155A-G. As visible in FIG. 5, each rim protrusion of the plurality of rim protrusions 155A-G is hollow. Rim protrusions may function to aid in seating a lid of a container onto a body of a container. For example, each rim protrusion 155A-G may have a negative draft angle, and in this way, when used with a lid and a container, may function to provide a friction fit between the lid and outer insert 150, as described in greater detail elsewhere in the present disclosure.

As to the wall of outer insert 150, the wall includes a series of levels that progressively define a smaller periphery of the recessed portion until reaching base 180, as shown in FIGS. 5 and 6. An overall shape of the wall combined with base 180 may be similar to a cup shape, with a cup shaped cavity therein. Beginning from rim 152, the wall includes rim wall 159 that defines an initial opening into a cavity of outer insert 150, as shown in FIG. 6. In the depicted example, a maximal opening size of the cavity of outer insert 150 is defined by an inner surface of rim wall 159. At a base of rim wall 159 is rim wall step 160 that extends transversely to rim wall 159 in a radially inward direction. Moving further inward, the wall includes a plurality of wall levels including first wall 161, second wall 163, third wall 165, fourth wall 167, and fifth wall 169. Such wall levels, e.g., first through fifth walls 161, 163, 165, 167, 169 may also be referred to as wall portions throughout the present disclosure. Each wall is separated by a step that is oriented transverse to the respective walls. First step 162 separates first and second walls 161, 163, second step 164 separates second and third walls 163, 165, third step 166 separates third and fourth walls 165, 167, fourth step 168 separates fourth and fifth walls 167, 169 and fifth step 170 separates fifth wall 169 and a periphery of base 180.

Each wall level, including first through fifth walls 161, 163, 165, 167, 169, is shaped such that along a length direction of the wall, a radius of curvature of a surface of the wall (outer and inner surfaces) varies. This characteristic may be shared among each of the wall levels, though dimensions at each level may still vary due to the decreasing size of the walls closer to base 180. Accordingly, for the sake of brevity, a shape of the wall levels will be described with reference to first wall 161 as representative of each wall level. First wall 161 includes a first segment 161A, a second segment 161B and a third segment 161C. Each segment 161A-C is separated from the others by a line of inflection where a convex surface becomes a concave surface or vice versa. In this way, and as shown in FIG. 5, first segment 161A has a convex outer surface, second segment 161B has a concave outer surface and third segment 161C has a convex outer surface. These characteristics result in a wall that has a varying radius of curvature along its length. Forming outer insert 150 with walls having the above-described shape characteristics is advantageous in that it may be used to control and render predictable where bending will occur on the recessed portion of the outer insert 150 when the outer insert is subject to forces sufficient to cause deformation of outer insert 150. One reason for this is that first through fifth walls 161, 163, 165, 167, 169 have non-uniform geometry along their length, e.g., each has a varying curved contour that disrupts what would otherwise be a surface with a single radius of curvature, and this compensates for any variability in material thickness in the outer insert. In a design without such non-uniform geometry, there may be an increased risk that bending may occur in an unpredictable and undesirable manner in parts of the outer insert that are thin relative to others. Additionally, the shape of the walls advantageously provides a more uniform compression of outer insert 150 when outer insert 150 is subject to forces.

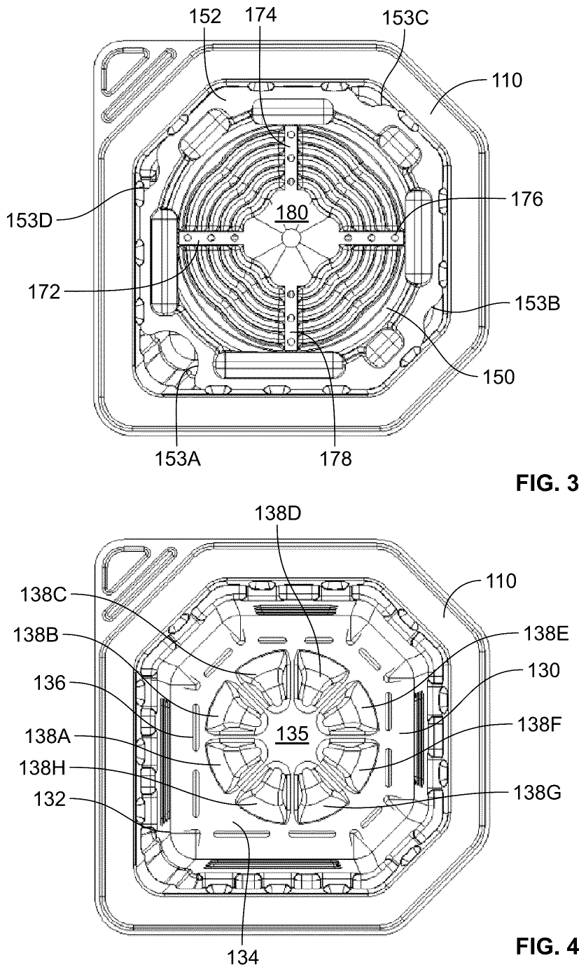

In the depicted embodiment, the wall also includes a plurality of radial protrusions 172, 174, 176, 178 that are raised relative to an outer surface of the walls 161, 163, 165, 167, 169. Each radial protrusion 172, 174, 176, 178 has a length dimension that extends from base 180 to rim 152 in a plane that bifurcates the enclosed shape of the wall and base 180 combination. Each radial protrusion may be equally spaced apart as shown in FIG. 3 to define an “X” or plus shape. In variations, it is envisioned that the number and location of such radial protrusions on the wall may vary from that shown. Each radial protrusion may include one or more openings therethrough, such as those shown in FIG. 5, where third radial protrusion 176 includes openings 186A-C and fourth radial protrusion 178 includes openings 188A-C. Radial protrusions are advantageous in that they provide additional rigidity and strength to outer insert 150 to withstand forces that may be applied thereto. Additionally, the radial protrusions 172, 174, 176, 178 on the recessed portion of outer insert 150 also form a break in the walls 161, 163, 165, 167, 169 and steps 162, 164, 166, 168. 170. Such break disrupts the recessed portion in planes corresponding to the wall surfaces and the step surfaces, and in this way, enhances a uniformity of deflection of the recessed portion when outer insert 150 is pressed against an object. In variations, the inclusion of additional radial protrusions may further increase the rigidity of the outer insert and may be used to influence a degree of uniformity of deformation of the recessed portion when subject to forces. Each radial protrusion may be hollow in the sense that a thickness of material in a wall portion (e.g., one of first through fifth walls 161, 163, 165, 167, 169) may be generally similar to a thickness of material in a radial protrusion (e.g., one of radial protrusions 172, 174, 176, 178). In this way, on an inner surface of the recessed portion as shown in FIG. 6, inner surfaces of radial protrusions also project relative to inner surfaces of walls 161, 163, 165, 167, 169 and steps 162, 164, 166, 168, 170.

With continued reference to outer insert 150, at a base of the wall remote from rim 152 is base 180. Base 180 as depicted includes a base outer surface 181 that may be generally smooth and have a curvature. As depicted, base outer surface 181 has a peripheral edge that generally follows a shape of the respective wall levels, as shown in FIG. 5. In this way, base 180 projects further from an apex of base 180 at the respective radial protrusions. As depicted, each radial protrusion 172, 174, 176, 178 extends from base 180 and has an outer surface flush with central outer base surface 181. An apex of the cavity of outer insert 150 may include apex opening 189, such opening extending through a thickness of base 180. This and the other openings through the wall are advantageous in that they allow for the passage of fluid through outer insert 150, which may facilitate certain sterilization procedures, for example.

In some examples, each of inner insert 130 and outer insert 150 may be made of polymeric materials, such as thermoplastic polyurethane. The materials used to fabricate the inserts have flexibility that allows for deformation of the inserts when subject to forces, such as those that occur when packaging an implant in a container using such inserts. Further, the material of the inserts, such as thermoplastic polyurethane, has elastic properties. This provides extra resistance, i.e. grip when an outer insert is held in place within a container by a lid on one side and presses against and deforms over an implant on an opposite side. The use of other elastically deformable materials to fabricate the inner and outer inserts is also envisioned.

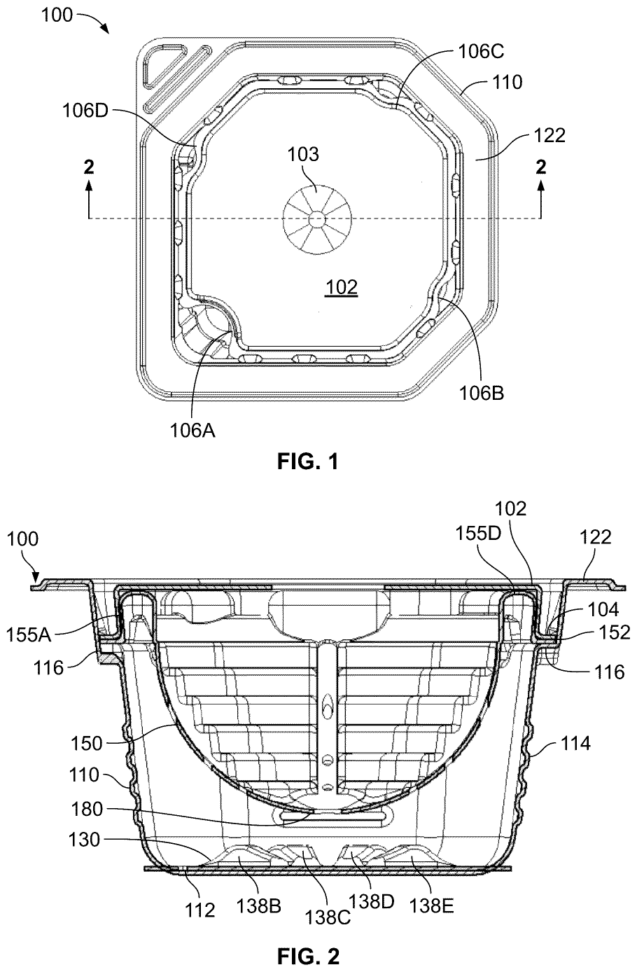

In some embodiments, a system including inner insert 130 and outer insert 150 may also include a container and a lid, such as container 110 and lid 102. In this manner, a packaging system 100 may include inner insert 130, outer insert 150, container 110 and lid 102, as shown in FIGS. 1-6.

Container 110 is shown in FIGS. 1-4 and includes a peripheral rim 122 with a recessed portion enclosed therein. A cavity of the recessed portion is defined by an inner sidewall 114 and an inner base surface 112, as shown in FIG. 2. Inner sidewall 114 also includes a receiving step 116 that may extend along an entire periphery of the cavity or alternatively may extend over one or more subportions of the periphery of the cavity. Receiving step 116 is sized and located along the sidewall of container 110 to receive outer insert 150 and lid 102 thereon, as shown in FIG. 2 (and FIG. 3 for outer insert 150). Inner sidewall 114 has a shape above receiving step 116 such that a peripheral edge of rim 152 of outer insert 150 is complementary to inner sidewall 114, as shown in FIG. 3. However, at peripheral recesses 153A-D along rim 152, there is a gap between outer insert 150 and inner sidewall 114, allowing for fluid communication therebetween. Further, at the base of the recessed portion remote from the rim of container 110, inner base surface 112 is sized and shaped to receive inner insert 130, as shown in FIGS. 2 and 4.

Lid 102 includes an outer periphery shaped to fit within inner sidewall 114 of container 110, as shown in FIGS. 1 and 2. The outer periphery of lid 102A is defined by a lid rim 104 that is recessed relative to a central portion of the lid internal to lid rim 104, as shown in FIG. 2. Outer edge of lid rim 104 generally complements a portion of inner sidewall 114 of container 110 that is above receiving step 116, though includes peripheral recesses 106A-D that are complementary to peripheral recesses 153A-D of outer insert 150. As noted above, this ensures fluid communication into and out of a cavity defined by lid 102 attached to container 110. Lid 102 may be sized and may be made of materials with flexural properties so that lid 102 may be snap-fit, e.g., snap locked onto container 110 to maintain securement of lid 102 on the container until such container is removed through an affirmative act, such as by pulling the lid 102 off the container 110. Further, the lid 102, outer insert 150 and container 110 combination is such that the outer insert 150 may be sandwiched in between the lid 102 and the cavity of container 110 to be held in place by the lid 102 when the system is closed, i.e., the lid is secured to the container. In the depicted embodiment, lid 102 may include a lid opening 103, as shown in FIG. 1. Such opening 103 may further facilitate fluid communication into and out of the cavity within container 110. Containers contemplated by the present disclosure, such as container 110 and accompanying lid 102, may be made of any materials capable of storing sterilized objects. Further, the lid and/or container any of the contemplated embodiments may be rigid relative to the inner and outer inserts. In some examples, the lid may be made of a material having a higher modulus of elasticity than the inner and outer inserts.

It should be appreciated that inner insert 130 and outer insert 150 as described above are adapted to fit into container 110 and are also adapted to fit into other containers having a different volume than container 110. This characteristic of inserts 130, 150 is described in greater detail elsewhere in the present disclosure.

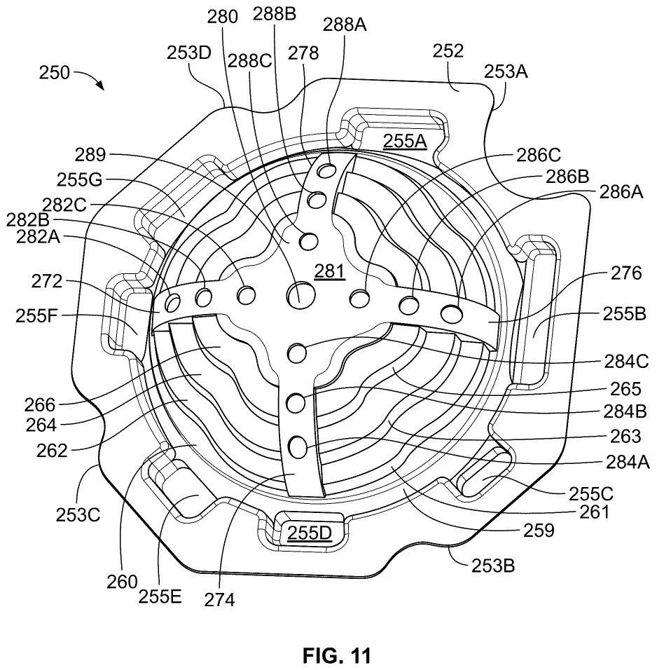

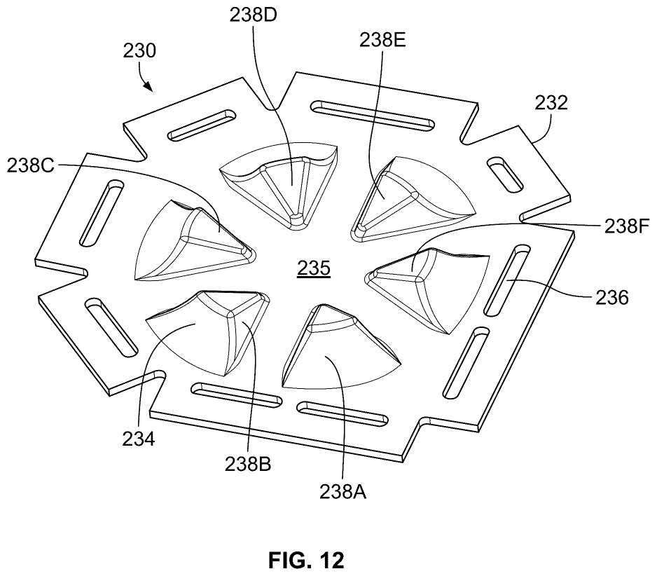

In another embodiment, a system includes an inner insert 230 and an outer insert 250 as shown in FIGS. 11 and 12. Unless otherwise indicated, for inner insert 230, like reference numerals refer to like elements of inner insert 130 as shown in FIGS. 2 and 4, but within the 200 series of numerals. Similarly, unless otherwise indicated, for outer insert 250, like reference numerals refer to like elements of inner insert 150 as shown in FIGS. 2, 3 and 5-6, but within the 200 series of numerals.

Inner insert 230 is shown in FIG. 12 and includes an upper surface 234 with a peripheral edge 232. Inner insert 230 also defines a plurality of slits 236 located adjacent to and generally parallel to peripheral edge 232. Further, peripheral edge 232 includes recesses between individual slits or sets of slits. Such recesses promote ease of folding portions of inner insert 230 along a line of one or more slits. Inner insert 230 also includes a plurality of support protrusions 238A-238F. Similar to inner insert 130, the plurality of support protrusions may be circumferentially disposed around a central region 235 of inner insert 230. A shape of each support protrusion may be such that an upper surface of each support protrusion rises in a radially outward direction from central region 235 and becomes wider until reaching a high point, from which it includes another surface sloped back to upper surface 234. A spacing between each support protrusion of plurality of support protrusions 238A-238F may be greater than that of plurality of support protrusions 138A-H of inner insert 130.

Outer insert 250 is shown in FIG. 11 and includes a rim 252 and a recessed portion extending from within the rim to define a cavity. The recessed portion includes a tapering wall that extends between rim 252 and base 280. As with outer insert 150, rim 252 of outer insert 250 includes a plurality of peripheral recesses 253A-253D and a plurality of rim protrusions 255A-255G. Along the tapering wall are a rim wall 259 extending inward from rim 252, followed by a plurality of wall levels 261, 263, 265, each separated by a respective step 260, 262, 264, 266. Each of first, second and third walls 261, 263, 265 has a similar wavy shape along its length as described for the walls of outer insert 150. Outer insert 250 also includes first, second, third and fourth radial protrusions 272, 274, 276, 278, each protruding relative to walls 261, 263, 265 as shown in FIG. 1. In the depicted embodiment, each radial protrusion is equally spaced apart. Along each radial protrusion are a series of openings, such as openings 282A, 282B, 282C for first radial protrusion 272. And, at the apex of the recessed portion there is apex opening 289 extending through base 280. Outer insert 250 shares in the advantages of outer insert 150.

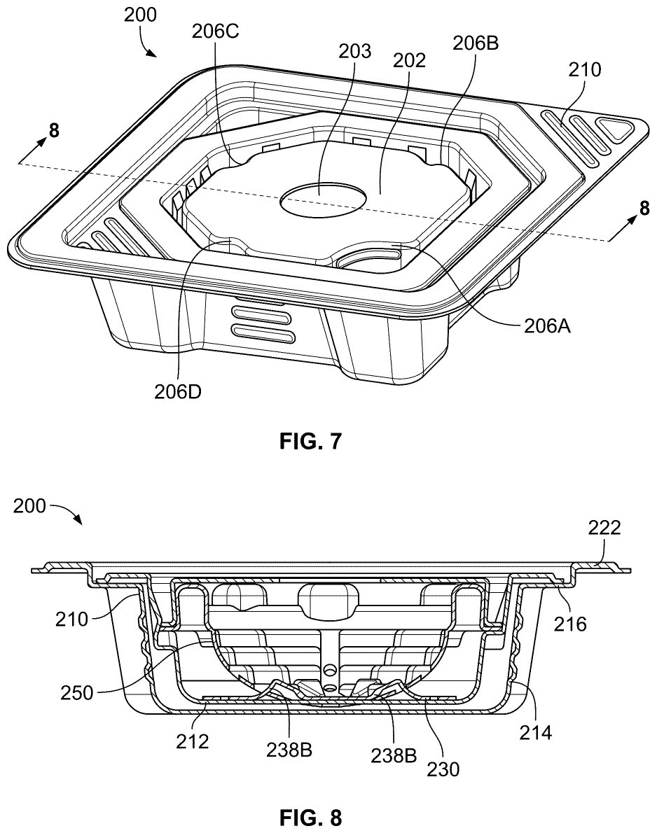

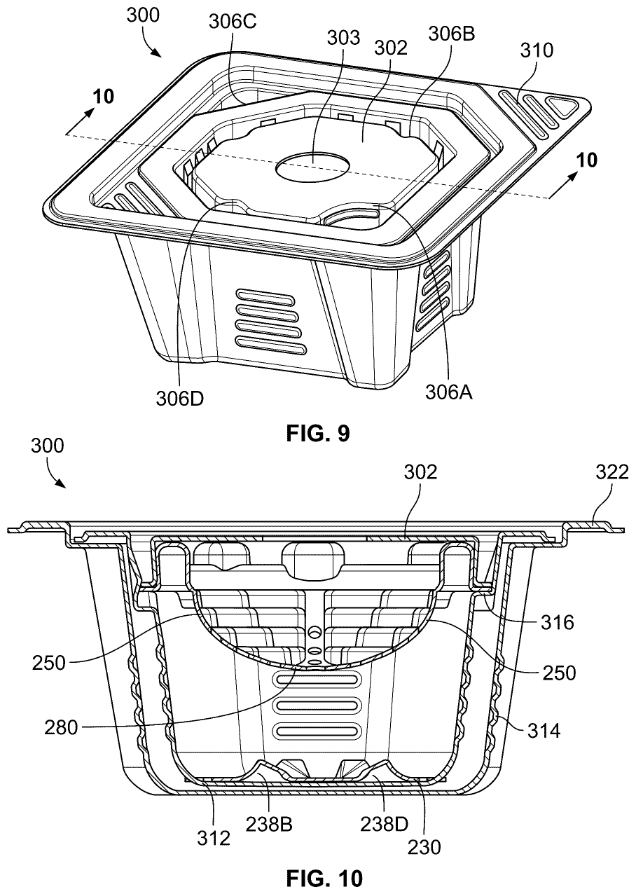

In further embodiments, inner and outer inserts 230, 250 may be part of a packaging system with a container, as similarly contemplated for inner and outer inserts 130, 150. In one embodiment, a packaging system 200 includes inner insert 230, outer insert 250, lid 202 and container 210, as shown in FIGS. 7 and 8. In yet another embodiment, a packaging system 300 includes inner insert 230, outer insert 250, lid 302 and container 310, as shown in FIGS. 9 and 10. Each of containers 210, 310 may include the same features as container 110, and numerals in the 200 and 300 series of numerals refer to like elements in the 100 series of numerals. Further, it should be appreciated that these depicted systems illustrate one example of how inner insert 230 and outer insert 250 are universally compatible with multiple container sizes, e.g., containers that have varying internal volumes. Specifically, inner insert 230 and outer insert 250 may be received in either container 210 or container 310 for securing an object within the respective container.

One or more of the inner insert, outer insert and container of the system may be varied in many ways. As to the inner insert, in some examples, the inner insert may have a different peripheral shape from that shown in FIGS. 4 and 12. Such peripheral shape may be any shape that complements a container into which the inner insert is to be received. Similarly, an overall surface area of an upper surface of the inner insert may vary. In some examples, a quantity of support protrusions may be one or more support protrusions. In some examples, a sloped receiving surface of the support protrusion may have shapes other than the generally triangular shape shown in FIGS. 4 and 12. For example, the sloped receiving surface of the support protrusion may be square, rectangular or trapezoidal. In still further examples, the plurality of support protrusions of the inner insert may collectively define a non-circular pattern. Such patterns may include square, rectangular, polygonal, oblong and ovular, among others.

With continued reference to variations of the system components, and with specific reference to the outer insert, in some examples, the outer insert may have a different peripheral shape from that shown in FIGS. 3 and 11. Such peripheral shape may be any shape that complements a container into which the outer insert is to be received. Similarly, an overall area defined by a peripheral edge of a rim of the outer insert may vary. In some examples, a contour of each wall along the recessed portion may vary from that shown in the depicted embodiments. For example, a wall segment between two radial protrusions may include two convex parts and two concave parts arranged in an alternating manner, i.e., convex, concave, convex, concave. In some examples, a segment may have five or more segments. An absolute radius of curvature of each segment and relative radii of curvature between segments may also vary from that shown in the depicted embodiments. In still further examples, one or more segments of a wall segment may be planar. In some examples, an outer insert may have any number of wall levels to define a recessed portion. For instance, one rim wall, one rim wall and one wall level, one rim wall and two wall levels, and so on. Similarly, a quantity of radial protrusions may vary from the four shown in FIG. 3. For example, an outer insert may have no radial protrusions or may have two, three, five, six, or more radial protrusions. Such radial protrusions may be equally spaced apart or at other spacings, such as irregular spacings. Further, a width of each radial protrusion may be less or greater relative to a size of outer insert relative to those in the depicted embodiments. An outer insert may also have any number of openings through a combination of the base and the radial protrusions.

With continued reference to variations of the system components, and with specific reference to the container, in some examples, the container may have a receiving feature different from the receiving step 116, 216, 316 shown in FIGS. 2, 8 and 10, respectively. For example, an inner wall surface of the container may be adapted to receive and engage with an outer insert and a lid. Further, a mechanism to facilitate securement of the lid onto the container may be through an adhesive, a male to female connection, or other engagement mechanism. A lid for use with a container may also vary from that shown. For example, a lid may have an outer periphery shaped to accommodate engagement features or an overall shape of a container. Lid, and similarly outer insert, may also have a different arrangement of peripheral recesses from that shown in the depicted embodiments. For example, a shape and/or quantity of such peripheral recesses may vary from that shown.

In each of the contemplated embodiments, a pair of inserts inclusive of an inner and outer insert may be adapted for receipt in a set of containers that may include two or more containers, where each container has a different internal volume capacity. In this way, the packaging system is advantageous in that it reduces the number of containers and container components necessary to store a set of implants or other objects relative to systems that do not utilize universal inserts.

In another aspect, insert components adapted for use with a container may be standalone devices. In some embodiments, an inner insert may be a standalone device adapted for use in a container to aid in the containment of an object. Such inner insert may be inner insert 130, 230, or any other inner insert contemplated by the present disclosure. Similarly, an outer insert may be a standalone device adapted for use in a container to aid in the containment of an object. Such outer insert may be outer insert 150, 250, or any other outer insert contemplated by the present disclosure.

In one aspect, the present disclosure relates to a kit for packaging an object. In some embodiments, a kit may include an inner insert, an outer insert and a container with complementary lid, where the inner and outer inserts are adapted to be received in the container. In some embodiments, a kit may include an inner insert, an outer insert and two or more containers, each optionally having complementary lids, where the inner and outer inserts are adapted to be received in each of the two or more containers. For instance, a kit may include inner and outer inserts, and three, four or more containers. In some variations of such kits, the containers may have the same rim geometry, but may have recessed portions with varying depths. For example, a kit may include inner insert 230 (FIG. 12), outer insert 250 (FIG. 11), lid 202, container 210 (FIGS. 7-8), lid 302 and container 310 (FIGS. 9-10). In some examples where a kit includes two or more containers, a kit may include three containers where each container has a recessed portion defining a cavity with a volume different from the other containers, e.g., small, medium and large containers. Other examples of a kit may include four, five or more containers where each has a different volume. A difference in the volume of each container may be based on a difference in a depth of each container where the depth is measured from a rim of the container to a base of the container.

In some embodiments, a kit may include two or more sets of inner and outer inserts. In some of these embodiments, at least two of the sets of inner and outer inserts may include inner and outer inserts that are adapted for receipt in different types of containers. For instance, a first set of inner and outer inserts may be adapted for use with a first set of containers having different container sizes and a second set of inner and outer inserts may be adapted for use with a second set of containers having different container sizes, where the containers of the second set are different from the containers of the first set. In one specific example of such a kit, the first set of inserts may be adapted for use with a first set of containers having a square shaped rim while the second set of inserts may be adapted for use with a second set of containers having a circular shaped rim. In another specific example, the first set of inserts may be adapted for use with a first set of containers having a square shaped rim with a first perimeter dimension while the second set of inserts may be adapted for use with a second set of containers having a square shaped rim with a second perimeter dimension different from the first perimeter dimension.

Further, in any one of the above embodiments, the kit or individual items and combinations thereof may be disposed within a kit package or a plurality of kit packages. Kit packages should be understood as distinguishable from a container and are adapted for holding the contemplated containers and other components. For example, all of the items of the kit may be disposed within a single kit package. In another example, all of the inner inserts may be disposed in one kit package, all of the outer inserts disposed in another kit package, and where applicable, all of the containers disposed in yet another kit package. The items included in the kit may also be individually packaged in individual kit packages. For example, each container may be in its own kit package. Packaging each item in the kit separately or in different combinations may improve the sterility of the items in preparation for use in storing or transporting objects such as medical implants. In any of the above embodiments, a kit may further include an instruction manual with an explanation of details relating to the contents of the kit including instructions for use of the contents.

In another aspect, the present disclosure relates to a method of fabricating inner inserts, outer inserts and/or containers usable therewith. In some embodiments, injection molding may be used to fabricate one or more components of a packaging system. In some embodiments, machining techniques may be used to fabricate one or more components of a packaging system. In still further embodiments, additive layer manufacturing may be used to form one or more components of a packaging system. Examples of additive layer manufacturing techniques that may be utilized include Fused Deposition Modelling (“FDM”), Shape Deposition Manufacturing (“SDM”), Selective Laser Power Processing (“SLPP”), various forms of laser sintering including Selective Laser Sintering (“SLS’), Selective Laser Melting (“SLM”), Selective Heat Sintering (“SHS”), Electron Beam Melting (“EBM”), material jetting, binder jetting, or the like. Additional details of exemplary additive manufacturing methods are described in U.S. Pat. Nos. 7,537,664, 8,590,157, 8,728,387, 9,180,010 and 9,456,901, the disclosures of which are hereby incorporated by reference herein in their entireties.

In another aspect, the present disclosure relates to a method of packaging an object within a container using an inner insert and an outer insert. As noted elsewhere in the present disclosure, it should be appreciated that a wide variety of objects may be held by a container through the performance of methods as contemplated by the present disclosure. In some examples, an object is a medical implant, such as an acetabular cup. A medical implant may also be referred to as simply an implant, and the methods below, while making reference to one or more implants, should be understood to apply to any number of different objects. References to an implant are made solely for the sake of brevity.

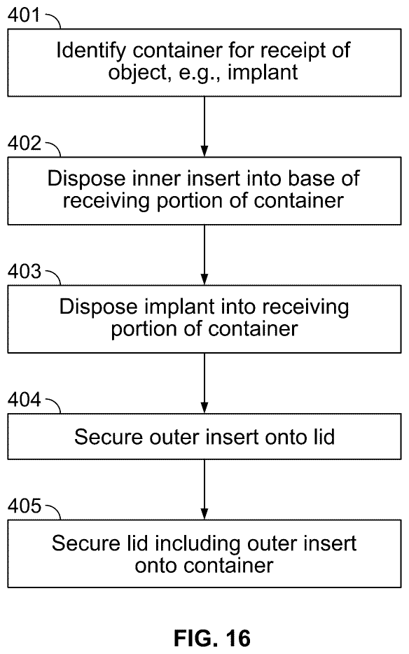

In some embodiments, a method of securing an implant into a container is performed through a series of steps as shown in FIG. 16. In a first step 401, a container is identified for receipt of an implant. In some examples, this may simply involve use of a single available container, or, in other examples, this step may optionally entail selection of a container from among a set of containers. Where a set of containers are available, the implant may be compared to the containers to evaluate which container is best suited to store the implant. Considerations for such determination may include whether the container has sufficient volume to hold the implant and whether the container is small enough so that when the implant is disposed in the container, the inner and outer inserts will press against the implant, as described in greater detail below. For purposes of illustration, an implant with a height beyond the upper limit of a first container may be checked against a container one size larger within a set of containers, and then against another container one size larger than that as needed until a container with sufficient depth to hold the implant is identified. In some examples, a maximum implant height for use with a specific container size is the minimum implant height for use with a specific container size that is one size larger. No matter the container used, the same universal inner and outer inserts may be used, with the outer insert deforming when compressed within the container when the implant is disposed therein, as described in greater detail below.

Once a container is selected, a second step 402 includes disposing an inner insert onto a base of the container. For example, inner insert 230 is disposed onto base 212, 312. The inner insert is disposed so that a peripheral edge of the inner insert is aligned with a periphery of the base of the container to ensure a proper fit. In some variations of the method, the inner insert and base may have complementary features to further secure the inner insert to the base, such as a respective tab and slot. Further, in some variations, one or more peripheral regions of the inner insert may be folded to obtain an optimal fit of the inner insert in the container. For instance, folding of the one or more peripheral regions may be performed when a surface area of the inner insert is greater than a surface area of a bottom surface of the container.

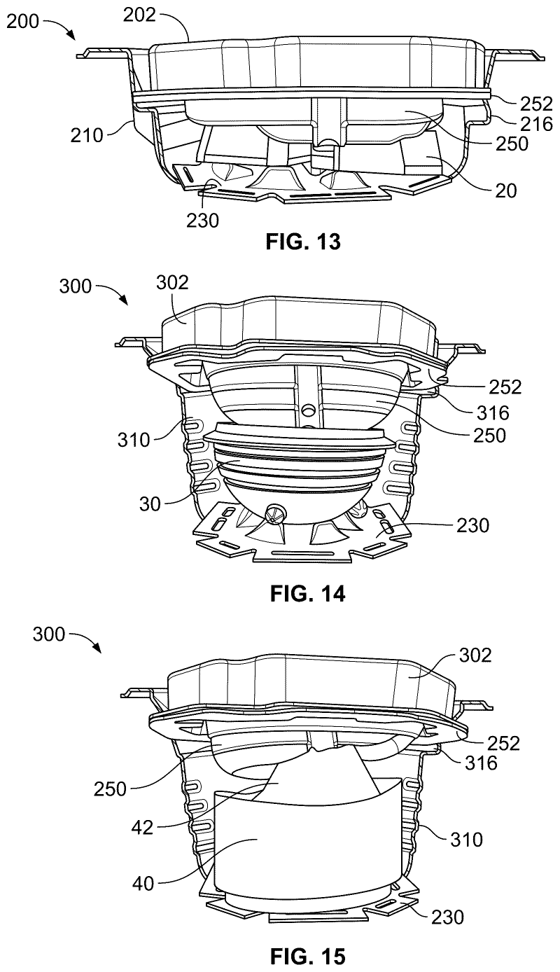

In a third step 403, the implant is disposed into a cavity of the container over the inner insert, e.g., inner insert 230. A portion of the implant presses against one or more support protrusions of the plurality of support protrusions of the inner insert. Such interfacing with the support protrusions provides grip that serves to resist movement between the implant and the plurality of support protrusions when the method is complete and the implant is fully contained. Examples of an implant placed on an inner insert are shown in FIGS. 13-15, where implants 20, 30, 40 are disposed on respective inner inserts 230.

In a fourth step 404, an outer insert is secured to a lid. In some examples, an outer insert, such as outer insert 150, is pressed into lid 102. Specifically, and as shown in FIG. 2, rim protrusions 155A-G are pressed into a cavity on an underside of lid 102. Because of the negative draft angle of the rim protrusions, the rim protrusions press outward against lid 102 to retain outer insert 150 within lid 102 via a friction fit. The same principles may be applied to secure outer insert 250 into lid 202, 302, or to secure any other outer insert as contemplated by the present disclosure. In variations, a system used in a method may include a lid and outer insert combination with other features to secure the outer insert to the lid.

In a fifth step 405, the lid with the outer insert secured therein is engaged, e.g., pressed onto the container to enclose the contents, e.g., implant. As the lid is pressed onto a receiving step of the container, e.g., receiving step 116 or another engagement surface, the outer insert deforms as it presses against the implant already seated on the inner insert. For example, as shown in FIGS. 13-15, outer insert 250 may be pressed onto implant 20, 30, 40 sufficiently to bring a rim 252 of outer insert 250 onto a receiving step of the container, while in the process outer insert 250 deforms against the implant. The deformation is based on portions of the outer insert pressing against regions of the implant, and in this way, such deformation causes the outer insert to have a closer fit to complementary surfaces of the implant. Further, outer insert 250 provides additional resistance to hold the implant in place in that after deforming over the implant, the elastic properties of outer insert 250 cause outer insert to press against the implant to enhance the grip of the outer insert 250 against the implant. Put another way, the elastic properties of outer insert 250 urge the outer insert 250 to revert to its undeformed shape, but the presence of the implant prevents the outer insert from doing so. The process may also be described as the deformation of the flexible material of the outer insert so that it conforms to a surface shape of an adjacent surface of the implant, while the flexible material continues to apply force against the implant after deformation due to the elastic properties of the outer insert. This conforming deformation occurs irrespective of the size, shape or surface finish of the implant. Examples of the described deformation of the outer insert over an implant are shown in FIG. 13 where central outer surfaces of outer insert 250 are deformed over the elongate shape of implant 20, and in FIG. 15 where a central outer surface of outer insert 250 is deformed over a central protrusion 42 of implant 40.

The above steps complete the securement of the implant inside the container. When secured within the container, the outer insert is compressed and deformed to conform around the implant and the outer and inner insert together secure the implant against movement within the container. Additionally, the outer and inner inserts absorb vibration and shock forces that the container may be subject to during its lifecycle, which in turn reduces the risk of damage to the implant. Further, the inner and outer inserts have a high abrasion resistance and thus minimal particulate is generated within a container holding an implant even when the container is subject to forces. FIGS. 13-15 show implants 20, 30, 40 secured in containers with lids set into place thereon. One part of the recessed portion of the respective containers 200 (FIGS. 13), 300 (FIG. 14), 300 (FIG. 15) is cut out in the images for illustration purposes only so that the components of the applicable packaging systems 200, 300 are visible. The method described above may be performed using inner insert 130 and outer insert 150, inner insert 230 and outer insert 250, or any other set of inserts as contemplated by the present disclosure.

In still further embodiments, the steps of the above-described methods may be performed in a different order. For example, step 404 may be performed earlier in the method. In still further embodiments, a method may be performed that includes one or more of the contemplated method steps. For example, the method may optionally proceed directly from step 401 to step 403. In such example, the containers used may include a bottom surface with a surface contour adapted to receive an object. In another embodiment, a method of removing an implant is contemplated. In such method, a lid and outer insert are removed from a container, and the implant is retrieved.

In another aspect, a method of sterilization is performed. In one embodiment, a sterilization method is performed with a packaging system such as one of packaging systems 100, 200, 300. With an implant disposed within the packaging system, fluid used in a sterilization process may be fed into the container 110, 210, 310 via openings defined by peripheral recesses 106A-D, 206A-D, 306A-D and peripheral recesses 153A-D, 253A-D and separately via fluid passage through lid opening 103, 203, 303 and openings 182A-C, 184A-C, 186A-C, 188A-C, 189, 282A-C, 284A-C, 286A-C, 288A-C, 289 in outer insert 150, 250. In other embodiments, outer insert may have a different arrangement of peripheral recesses and/or openings to facilitate the passage of fluid therethrough. Such fluid communication between areas external to the packaging system and the implant held therein is advantageous in that it allows for sterilization of implants while the implant is secured inside a closed container. Further, the contemplated packaging systems are advantageous in that the designs allow for fluid communication, e.g., gas flows, utilized in a wide variety of sterilization methods.

While the methods described in the present disclosure may be performed manually, some embodiments may employ robots to aid in the use of systems and kits as contemplated by the present disclosure. In one non-limiting example, a robot may be programmed to control placement of an inner insert and an outer insert into a container, along with placement of an object into the container. A robot may further be used to apply a lid to close the container. Similarly, a robot may also be used to retrieve an object already enclosed in a container. Robots may optionally be employed for use with any system or kit contemplated by the present disclosure. Examples of robotic systems that may be used include those described in U.S. Pat. Nos. 6,676,669; 7,892,243; 6,702,805; 6,723,106; 7,035,716; 6,757,582; 7,831,292; 8,010,180; 7,950,306; and 9,622,823; and U.S. Pat. App. Pub. Nos. 2010/0268249; 2010/0268250; 2010/0275718; and 2003/0005786, the disclosures of which are hereby incorporated by reference herein in their entirety.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A packaging device comprising:
a first insert including a rim enclosing a recessed portion, the recessed portion including a base and a wall extending between the rim and the base, the wall having a plurality of steps,
wherein the first insert is configured such that the rim is receivable on a surface of any one container from among a plurality of containers, and,
wherein the plurality of containers include a first container and a second container such that a first interior volume is defined between the first insert and a base of the first container when the first insert is received on the first container and a second interior volume is defined between the first insert and a base of the second container when the first insert is received on the second container, the first interior volume being different from the second interior volume.

2. The packaging device of claim 1, wherein each step of the plurality of steps is separated by a wall portion of a plurality of wall portions, each of the wall portions having a varying radius of curvature along its length.

3. The packaging device of claim 1, wherein a portion of the wall of the first insert that includes the plurality of steps is deformable when placed over and pressed against an object disposed in one container from among the plurality of containers.

4. The packaging device of claim 1, wherein the plurality of steps on the wall include edges having a varying radius of curvature along their respective lengths.

5. The packaging device of claim 1, wherein the first insert further comprises a plurality of radial protrusions extending from the base of the recessed portion to the rim along the wall of the recessed portion, the radial protrusions being on an outer surface of the recessed portion opposite an inner surface of the recessed portion that defines a cavity.

6. The packaging device of claim 1, further comprising the first container from among the plurality of containers.

7. A packaging system comprising:
the packaging device of claim 1; and
a second insert, the second insert being receivable on a bottom surface of any one container from among the plurality of containers.

8. The packaging system of claim 7, wherein the second insert includes a plurality of protrusions thereon, the plurality of protrusions being adapted to resist movement of an object disposed thereon.

9. A packaging system comprising:
a first insert having a recessed portion extending from an enclosed rim, the recessed portion having a central axis passing through a pole of the recessed portion and a center of an opening defined by the enclosed rim such that each step of a plurality of steps on the recessed portion extends circumferentially around the central axis; and
a second insert including a plurality of protrusions arranged around a central location of the second insert,
wherein the first and second inserts are configured to be received on respective first and second surfaces of a container,
wherein when the second insert is disposed on the second surface of the container and an object is disposed on the second insert, the disposal of the first insert onto the object causes the first insert to deform as the first insert is received on the first surface of the container, and
wherein when the first insert, the second insert, and the object are received in the container, the first and second inserts restrain the object from movement relative to the first and second inserts.

10. The packaging system of claim 9, wherein the first insert includes a plurality of openings therethrough.

11. The packaging system of claim 9, wherein the first insert includes a plurality of radially extending portions that extend from a base of the first insert toward the enclosed rim.

12. The packaging system of claim 11, wherein each radially extending portion of the plurality of radially extending portions is raised relative to the plurality of steps.

13. The packaging system of claim 11, wherein at least one radially extending portion of the plurality of radially extending portions includes an opening therethrough.

14. The packaging system of claim 9, wherein each step of the plurality of steps is separated by a wall portion of a plurality of wall portions, each wall portion having a varying radius of curvature along its length.

15. The packaging system of claim 14, wherein each step of the plurality of steps includes an outer surface configured to face the second insert when the first and second inserts are received in the container, the outer surface having a varying curvature along its length such that a centerline of the outer surface alternates between moving closer to and further from the central axis along its length.

16. The packaging system of claim 9, further comprising a lid configured to receive the first insert such that when the first insert is disposed onto the object in the container, the lid with the first insert secured thereto is secured to the container to enclose the object within the container.

17. An insert configured to restrain an object when disposed in a container, the insert comprising:
a rim; and
a recessed portion within the rim, the recessed portion including a wall and a base, the wall extending from the rim to the base,
wherein the wall includes a plurality of wall portions each separated by a step of a plurality of steps, each wall portion extending along the wall circumferentially around a central axis of a cavity defined by the wall and the base, the central axis passing through a center of the base and a center of an opening defined by the rim, at least one wall portion from among the plurality of wall portions having a an outer surface with a first segment having a convex surface and a second segment having a concave surface.

18. The insert of claim 17, wherein the wall further comprises a plurality of radial protrusions thereon, each radial protrusion of the plurality of radial protrusions being spaced apart from the others and extending from the base to the rim.

19. The insert of claim 18, wherein the plurality of radial protrusions includes four radial protrusions, each radial protrusion of the four radial protrusions being raised relative to the plurality of wall portions.

20. The insert of claim 17, wherein the rim includes an upper surface transverse to the central axis, the upper surface including a peripheral edge with a peripheral recess, the peripheral recess having a concave contour over a majority of its length.
